(19) United States
(12) Patent Application Publication
(10) Pub. No.: US 2006/0084931 A1
(43) Pub. Date: Apr. 20, 2006

(54) Multi-function medical device

(76) Inventor: Shao-Shih Huang, Taipei (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/967,352

(22) Filed: Oct. 19, 2004

(51) Int. Cl. A61M 1/00

(52) U.S. Cl. 604/317

(57) ABSTRACT

A multi-function medical device includes a compressor, a vacuum, an oxygen maker, a first electromagnetic valve, a second electromagnetic valve, a third electromagnetic valve. The compressor comprises an air inlet connected to the first electromagnetic valve and a filter, respectively. The first electromagnetic valve is then connected to the vacuum. An air outlet of the compressor is connected to the second electromagnetic valve and the third electromagnetic valve. The second electromagnetic valve is then connected to an air exhausting pipe. The third electromagnetic valve is connected to the oxygen maker and the air mixer, respectively. The device functions as an oxygen and vacuum machine, while the air mixer can be connected with different medical equipment for different purposes.

### FIELD OF THE INVETNION

This invention relates to a multi-function medical device, in particular, to a medical device connected with other medical equipment to provide multi-function capability.

### DESCRIPTION OF THE PRIOR ART

Medical devices are widely adapted in hospitals for medical purposes. There are various types of medical devices for different functions. In large and fully equipped hospitals, they are capable of purchase and store at their wards or emergency rooms. But, in a smaller hospital or in a private clinic, each device requires an extra budget and a larger space to store. Therefore, in many incidents, the cause is due to equipment shortage.

Therefore, the inventor derives the present device, which is a single medical device and expendable to perform multi-function medical purposes.

### SUMMARY OF THE INVENTION

It is the primary objective of the present invention to provide a multi-function medical device, which saves storage space.

It is another objective of the present invention to provide a multi-function medical device, which is expandable to operate different medical machines.

It is a further objective of the present invention to provide a multi-function medical device, which saves cost.

### BRIEF DESCRIPTION OF THE INVENTION

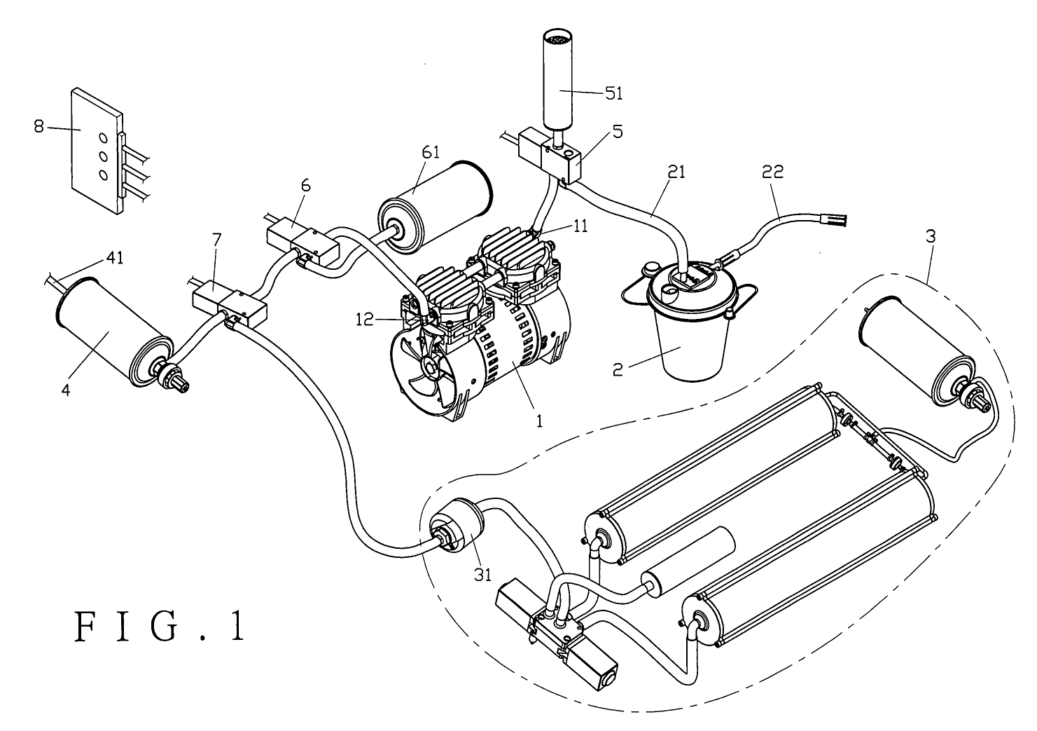

FIG. 1 is an exploded view of the present invention; and

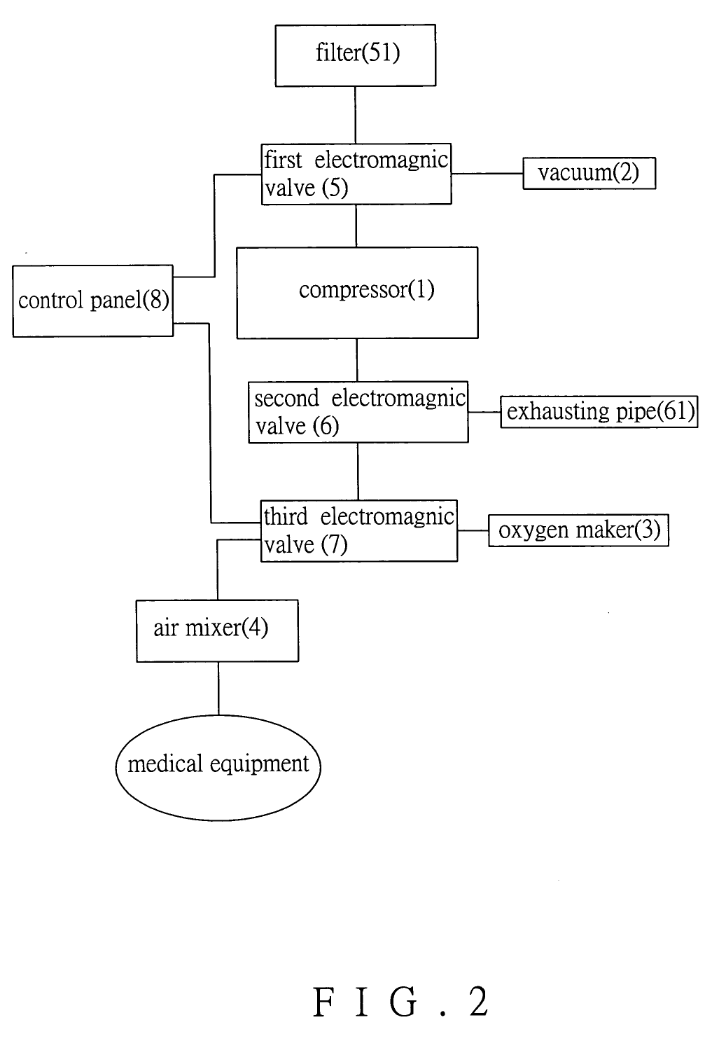

FIG. 2 is a flow chart diagram showing the operation of the present invention.

### DETAILED DESCRIPTION OF THE PREFERRED EMBODIMENT

As shown in FIGS. 1 and 2, a preferred embodiment of the present invention comprises a compressor 1, a vacuum 2, an oxygen maker 3, an air mixer 4, a first electromagnetic valve 5, a second electromagnetic valve 6, a third electromagnetic valve 7, a control panel 8, a filter 51 and an air exhausting pipe 61.

The compressor 1 includes an air inlet 11 and an air outlet 12. The air inlet 11 is connected to the first electromagnetic valve 5. The first electromagnetic valve 5 is connected to the vacuum 2 through a pipe 21 and to the filter 51, respectively. The vacuum 2 functions as a sputum sucking machine in this embodiment.

The air outlet 12 of the compressor 1 is connected to the second electromagnetic valve 6 and the third electromagnetic valve 7 through the second electromagnetic valve 6. The second electromagnetic valve 6 is connected to the air exhausting pipe 61. The third electromagnetic valve 7 is connected to the air mixer 4 and the oxygen maker 3 through a filter 31, respectively. The air mixer 4 has a pipe 41 extending outwardly to be connected to other medical equipment for other purposes. All the first electromagnetic valve 5, the second electromagnetic valve 6, and the third electromagnetic valve 7 are controlled by the control panel 8.

Other than the vacuum 2 and the oxygen maker 3, this device may be connected to other medical machine through the air mixer 4 and operate them for different medical purposes.

When an operator wants to suck sputum out of a patient, the control panel 8 is operated to shut down the filter 51, so that the compressor 1 is direct connected to the vacuum 2. When the compressor 1 is activated, objects, such as sputum, will be sucked out by the compressor 1 through a sucking tube 22 of the vacuum 2.

To operate the device as an oxygen machine, the control panel 8 is switched to activate the second electromagnetic valve 6 and the third electromagnetic valve 7. The oxygen maker 3 is connected to the third electromagnetic valve 7, by activating the compressor 1. Air goes through the filter 51 and the first electromagnetic valve 5 to the compressor 1, and then goes through the second electromagnetic valve 6, the third electromagnetic valve 7 and the filter 31 to the oxygen maker 3 and is supplied to the patient.

Further, if it is desired to connect the present invention to other medical equipment, the medical equipment is connected to the air mixer 4 through the tube 41. The control panel 8 is operated to activate the third electromagnetic valve 7. Thus air goes through the filter 51 into the first electromagnetic valve 5, the second electromagnetic valve 6, and the third electromagnetic valve 7, and enters into the air mixer 4. The alternative medical equipment, such as sleeping machine or sprayer will be activated.

From the above description, it is understood that any medical equipment or machine operated by air can be worked with this invention to reach medical purposes.

I claim:

1. A multi-function medical device comprising a compressor, a vacuum, an oxygen maker, a first electromagnetic valve, a second electromagnetic valve, a third electromagnetic valve, a filter, an air exhausting pipe and an air mixer;
said compressor comprising an air inlet and an air outlet, said air inlet being connected to said first electromagnetic valve and said filter respectively, said first electromagnetic valve being connected to said vacuum, said air outlet of said compressor being connected to said second electromagnetic valve and then to said third electromagnetic valve, said second electromagnetic valve being connected to said air exhausting pipe, said third electromagnetic valve being connected to said oxygen maker and said air mixer respectively, said air mixer having a pipe.

2. The multi-function medical device, as recited in claim 1, wherein the device is provided with a control panel to switch said first electromagnetic valve, said second electromagnetic valve, and said third electromagnetic valve.

3. The multi-function medical device, as recited in claim 1, wherein another filter is provided between said third electromagnetic valve and said oxygen maker.

4. The multi-function medical device, as recited in claim 1, wherein said vacuum is a sputum sucking machine.
